(19) United States
(12) Patent Application Publication
(10) Pub. No.: US 2007/0154516 A1
(43) Pub. Date: Jul. 5, 2007

(54) DRUG DELIVERY SYSTEM

(75) Inventors: Jonathan Bortz, St. Louis, MO (US); R. Saul Levinson, Chesterfield, MO (US)

Correspondence Address: HARNESS, DICKEY, & PIERCE, P.L.C, 7700 BONHOMME, STE 400, ST. LOUIS, MO, 63105, US

(73) Assignee: DrugTech Corporation, Wilmington, DE (US)

(21) Appl. No.: 11/617,199

(22) Filed: Dec. 28, 2006

Related U.S. Application Data
(60) Provisional application No. 60/756,804, filed on Jan. 5, 2006.

(51) Int. Cl. A61K 31/7048; A61K 31/7034; A61K 31/496; A61K 31/43; A61K 31/545

(52) U.S. Cl. 424/423; 514/152; 514/156; 514/35; 514/37; 514/39; 514/28; 514/29; 514/192; 514/200; 514/254.07; 514/397; 514/383

(57) ABSTRACT

A pharmaceutical composition comprises a first active (e.g., antibacterial) agent and a second (e.g., antifungal) active agent, and comprises a component that is adapted for bioadhesion to a vulvovaginal surface. The composition provides differential release of the active agents at such a surface, wherein the second active agent exhibits a release profile that is substantially delayed, extended and/or inverted relative to the release profile of the first active agent.

This application claims the benefit of U.S. Provisional patent Application Ser. No. 60/756,804, filed on Jan. 5, 2006, the entire disclosure of which is incorporated by reference herein. This application contains subject matter that is related to concurrently filed U.S. application Ser. No. ______, titled “Composition and method of use thereof”, and to U.S. application Ser. No. 11/326,979, filed on Jan. 5, 2006, the entire disclosure of each of which is incorporated by reference herein.

### FIELD OF THE INVENTION

The present invention relates to pharmaceutical compositions suitable for vaginal delivery of a combination of at least two active agents. The invention further relates to therapeutic methods of use of such compositions in women having conditions of the vulvovaginal system wherein such a combination of active agents is indicated.

### BACKGROUND OF THE INVENTION

Combination therapy involving two or more active agents is indicated in a number of conditions (including disorders, diseases and syndromes) affecting the lower urogenital tract or vulvovaginal system of female patients. In one scenario, such a condition can have multifactorial etiology, in which active agents having different modes of action can address more than one underlying cause. In another scenario, the condition can have a single underlying cause, but is treatable with one active agent that relieves symptoms and another active agent that attacks the underlying cause. In yet another scenario, the patient exhibits two or more conditions that are more or less independent of one another in their etiology, but that, when superimposed on one another, can present a more serious challenge to the health of the patient than either condition alone. In a still further scenario, the presence of a first condition can increase susceptibility of the patient to a second condition, and combination therapy is indicated to treat the first condition while preventing or reducing risk of the second. Yet further scenarios wherein a combination of two or more drugs is indicated will be readily envisioned by one of skill in the art.

An illustrative and all too common condition that can be responsive to combination therapy is infective vaginitis. Infective vaginitis covers a range of conditions involving microbial infection of the vagina, and inflammation associated therewith, that sometimes extends to the vulva, It accounts for an estimated 15 million physician office visits a year in the U.S., and with availability of over-the-counter remedies particularly for candidal infections, many additional cases are medicated without professional diagnosis.

Agents of infection implicated in vaginitis include:
(a) fungi, more particularly yeasts, especially Candida spp. including one or more of C. albicans, C. dubliniensis, C. glabrata, C. kefyr, C. krusei, C. lusitaniae, C. neoformans C. parasilopsis and C. tropicalis, of which the most common is C. albicans;
(b) bacteria, commonly a variety of species including one or more of Bacteroides spp., Gardnerella vaginalis, Mobiluncus spp., Mycoplasma hominis and Peplostreptococcus spp., most commonly with G. vaginalis predominating; and
(c) protozoa, especially Trichomonas vaginalis.

Candidal infections, herein referred to collectively as vulvovaginal candidiasis (VVC), are the best known cause of vaginitis and are believed to affect about 75% of women at least once during their lifetime. VVC is generally not sexually transmitted. Bacterial vaginosis (BV), a collective term used herein for vaginal or vulvovaginal conditions caused by bacterial infection, is generally considered a sexually transmitted disease although other modes of transmission can occur. Symptoms of VVC and BV include irritation (manifesting, for example, as redness, burning and/or itching), dyspareunia and abnormal discharge, which in the case of BV tends to have a fishy odor. Other diagnostic criteria include a vaginal pH lower than about 4.7 in VVC, or higher than about 4.7 in BV, and presence of “clue cells” (epithelial cells having a granular appearance) in BV.

VVC is typically a nuisance, often very troubling to the patient but relatively rarely implicated in development of more serious or life-threatening conditions. On the other hand, BV, if untreated, can lead to serious conditions, such as cervicitis, pelvic inflammatory disease, cervical dysplasia, urinary tract infections, postoperative infections, increased susceptibility to viral infection including HIV and HSV-2, and, in pregnant women, premature birth, preterm rupture of membranes, intra-amniotic fluid infection, preterm labor and postpartum endometritis.

Bacterial and candidal infections can coexist. Mixed bacterial and candidal (herein “BV/VVC”) infection occurs in up to about one-fifth of vaginitis cases. For example, Redondo-Lopez et al. (1990), Sex. Transm. Dis. 17(1):51-53, reported that in 132 episodes of symptomatic vaginitis in 35 patients with recurring symptoms, 15% were found to involve a mixed BV/VVC infection.

In another study, Ferris et al. (2002), Obstet. Gynecol. 99(3):419-425, reported that of 95 women who were about to treat themselves for VVC, 34% were confirmed to have VVC alone, 19% had BV alone, and 19% had a mixed BV/VVC infection.

A significant problem is that such mixed infections are underdiagnosed, and self-medication or prescribed treatment occurs as if for flugal or bacterial infection alone. Both fungi such as Candida albicans and bacteria such as Gardnerella vaginalis are opportunistic pathogens, therefore in case of a mixed infection removal of one can lead to rapid population growth of the other. Thus, for example, a mixed BV/VVC infection treated topically only with an antifungal agent such as butoconazole can quickly become a serious BV infection, which then requires follow-up antibacterial treatment, either as a further topical application or as systemic (e.g., oral antibiotic) therapy. Implications of such misdiagnosis can be nontrivial, especially considering the serious conditions to which BV can lead if untreated.

Thus a need exists in the art for a medicament and method of use thereof that conveniently and effectively treats BV and mixed BV/VVC infections. More broadly, a need exists for a convenient method of delivering a combination of two or more active agents for treatment of a vulvovaginal condition in a way that differentially times the delivery of each agent so as to maximize effectiveness and/or safety profile of the combination. Such differential timing can reflect a need, for example, to address one causal factor before another, or to simultaneously address an acute and a chronic condition, or to address first the symptoms and then the underlying cause of a condition, or, in the case of a mixed BV/VVC infection, to control a dominant bacterial (e.g., Gardnerella vaginalis) population and prevent a subsequent explosion of fungal (e.g., candidal) population in response to removal of the bacterial pathogens. Differential timing can also provide a means of reducing adverse side-effects of combination therapy, by avoiding simultaneous heavy exposure of tissues to two or more active agents. Combination therapies providing differential timing of drug delivery have typically involved sequential administration of active agents, for example topical administration of an antifungal agent followed by topical or systemic administration of an antibacterial agent as mentioned above.

A medical regimen involving multiple sequential administration of different active agents, often by different routes, can be complex and difficult for the patient to adhere to. A more convenient regimen, particularly one that can be satisfied by a single administration, would enhance patient compliance and thereby increase probability of a successful clinical outcome.

U.S. Pat. No. 4,551,148 to Riley et al. proposes a controlled release system for vaginal drug delivery, comprising unit cells having a nonlipoidal internal phase and a lipoidal continuous external phase. An active agent is present at least in the internal phase.

U.S. Pat. No. 5,266,329 to Riley proposes such a vaginal delivery system having an antifungal imidazole, exemplified by metronidazole, as the active agent.

Thompson & Levinson (2002), Drug Delivery Systems & Sciences 2(1), 17-19, describe a bioadhesive topical drug delivery system known therein as the VagiSite system as a high internal phase ratio water-in-oil emulsion system, providing a delivery platform for administration of active drug entities in the vaginal cavity. They disclose that the VagiSite system is incorporated in Gynazole-1® antifungal vaginal cream, which contains 2% by weight butoconazole nitrate.

U.S. Patent Application Publication No. 2003/0180366 of Kirschner et al. discloses a composition suitable for vaginal drug delivery, comprising an essentially pH neutral emulsion having an internal water-soluble phase and an external water-insoluble phase, wherein the internal phase comprises an acidic buffered phase comprising a drug, which can illustratively be an antifungal agent or an antibacterial agent. Example I therein provides such a composition comprising the antibacterial agent metronidazole in an amount of 0.75% by weight. Example II therein provides such a composition comprising the antibacterial agent clindamycin phosphate in an amount of 2.8% by weight.

U.S. Pat. No. 5,055,303 to Riley describes a solid composition, for example a suppository, comprising a water-in-oil emulsion that can carry an active agent. The composition is stated to be suitable for insertion into a body orifice and to melt at body temperature to form a cream having controlled release and bioadherent properties.

U.S. Pat. No. 6,316,011 to Ron et al. describes a reversibly gelling polymer composition having bioadhesive or mucoadhesive properties, said to be useful inter alia for delivery of drugs to a vaginal or rectal cavity.

U.S. Pat. No. 6,423,307 to Saettone et al. describes a mucoadhesive complex of polycarbophil with an imidazole or triazole active agent, said to be useful as a sustained-release antifungal preparation for vaginal administration.

International Patent Publication No. WO 02/03896 mentions inter alia a composition comprising a lipophilic or hydrophilic carrier and a mucoadhesive agent said to be useful for intravaginal delivery of an antifungal, antibacterial, antiviral, trichomonicidal or parasiticidal agent.

International Patent Publication No. WO 03/000224 relates to a composition comprising lactic acid and chitosan, said to be adhesive to a vaginal mucosa and to be useful in treatment of bacterial vaginosis and for restoring a physiological flora of lactobacilli.

Wang & Lee (2002), Contraception 66:281-287, evaluated a polymer gel formulation as a vaginal delivery system for microbicidal agents.

Gavini et al. (2002), AAPS PharmSci 2002, 3(3) article 20, 7 pp. (http://www.aapspharmsci.org) proposed a chitosan-based mucoadhesive vaginal delivery system for controlled release of the antimicrobial drug acriflavine.

Karasulu et al. (2002), J. Microencapsulation 19(3):357-362, described preparation of effervescent vaginal tablets containing the antifungal drug ketoconazole in microencapsulated form, using carboxymethylcellulose as a bioadhesive coating.

U.S. Patent Application Publication No. 2003/0091540 of Ahmad et al. relates to an ointment, said to be useful for delivery of an antifungal or antibacterial agent to the vaginal cavity. The ointment can have a bioadhesive agent to help promote adhesion to the mucosa.

U.S. Patent Application Publication No. 2003/0219472 of Pauletti et al. relates in part to a pharmaceutical composition stated therein to be useful for vaginal transmucosal delivery of a drug, and mentions that solubilization of the drug with an appropriate mucoadhesive agent can allow a prolonged contact of the drug with the mucosal surface, which is said to further enhance efficiency of delivery of the drug. Mention is made of bioadhesive microparticles in the form of a multiphase liquid or semi-solid preparation for vaginal delivery.

U.S. Patent Application Publication No. 2004/0151774 of Pauletti et al. describes a polymer foam or film composition suitable inter alia for delivery of a drug to a vaginal mucosa. The composition optionally provides controlled release of the drug and can include a mucoadhesive agent.

U.S. Patent Application Publication No. 2003/0091642 of Auzerie proposes a gel composition for application to a vaginal mucosa comprising a thermoreversibly gelling copolymer such as a poloxamer, a bioadhesive agent such as a carbomer, and at least one active agent in solution or suspension.

U.S. Patent Application Publication No. 2004/0234606 of Levine et al. proposes a composition for vaginal administration comprising a treating agent (the tocolytic drug terbutaline is exemplified) and a bioadhesive cross-linked water-swellable but water-insoluble polycarboxylic acid such as polycarbophil, designed to give controlled and prolonged release of the drug through the vaginal mucosa. Administration of the composition is said to achieve local tissue concentrations without detrimental blood levels.

U.S. Patent Application Publication No. 2003/0225034 of Floros et al. mentions that, for treatment of vaginitis, surfactant lipids can be administered in conjunction with one or more medications including antibiotics and antifungals. Examples of antibiotics said to be suitable include ampicillin, ceftriaxone, clindamycin, metronidazole and tetracycline. Examples of antiftngals said to be suitable include miconazole, clotrimazole, econazole, butoconazole, tioconazole and terconazole.

Özyurt et al. (2001) Int. J. Gynecol. Obstet. 74:35-43, evaluated efficacy of pessaries containing metronidazole 500 mg and miconazole nitrate 100 mg in candidal, bacterial, trichomonal and mixed vaginal infections. The pessaries were administered intravaginally twice daily for 7-14 days.

International Patent Publication No. WO 2004/096151 proposes inter alia an intravaginal drug delivery device providing controlled release of a plurality of drugs. The device is said to release the drugs in a substantially constant ratio over a prolonged period of time.

### SUMMARY OF THE INVENTION

There is now provided a pharmaceutical composition comprising a first active agent and a second active agent, the composition (i) comprising a component adapted for bioadhesion to a vulvovaginal surface, for example a vaginal mucosal surface, and (ii) providing differential release of the active agents at such a surface, wherein the second active agent exhibits a release profile that is substantially delayed and/or substantially extended relative to the release profile of the first active agent.

In one embodiment the first active agent is an antibacterial agent and the second active agent is an antifungal agent.

The composition illustratively has at least one nonlipoidal internal phase and at least one lipoidal external phase that is bioadhesive to the vulvovaginal surface. Such a composition is typically a water-in-oil emulsion and can illustratively be presented in a semi-solid form described in the pharmaceutical art as a cream.

There is further provided a vaginal drug delivery system comprising such a cream and an applicator to facilitate administration to a vaginal mucosal surface.

There is still further provided a method for treating a condition of the vulvovaginal system, for example a BV or mixed BV/VVC infection, for which a combination of a first active agent (e.g., an antibacterial agent) and a second active agent (e.g., an antifungal agent) is indicated, the method comprising administering a pharmaceutical composition as described herein to a vulvovaginal surface, for example a vaginal mucosal surface.

These and other embodiments are more fully described in the detailed description that follows.

### BRIEF DESCRIPTION OF THE DRAWINGS

FIG. 1 is a diagrammatic representation of relative release profiles of the first and second active agents in a composition of a first embodiment of the invention.

FIG. 2 is a diagrammatic representation of relative release profiles of the first and second active agents in a composition of a second embodiment of the invention.

FIG. 3 is a diagrammatic representation of relative release profiles of the first and second active agents in a composition of a third embodiment of the invention.

FIG. 4 is a diagrammatic representation of relative release profiles of the first and second active agents in a composition of a fourth embodiment of the invention.

### DETAILED DESCRIPTION

The term “vulvovaginal system” herein means the lower urogenital tract of a female subject, in particular the vaginal cavity and walls thereof and adjacent tissues of the cervix and urinary tract, together with the vulva. A “vulvovaginal surface” herein denotes any external or internal surface of the female genitalia, including mucosal surfaces in the vaginal cavity and nonmucosal surfaces of the vulva and immediately surrounding areas of skin. In some embodiments, a composition as described herein is more specifically adapted for application to a vaginal mucosal surface, and is bioadhesive, i.e., mucoadhesive, to such a surface.

Any known formulation system exhibiting bioadhesion to a vulvovaginal surface and capable of delivering thereto an active agent can be useful herein. Such systems include a variety of formulations described in International Patent Publication No. WO 2005/087270, incorporated herein by reference but not admitted to be prior art to the present invention.

Such systems further include, for example, those embodied in certain compositions generally described in above-referenced U.S. Pat. No. 6,316,011.

Such systems still further include those embodied in certain compositions generally described in above-referenced U.S. Pat. No. 6,423,307.

Such systems still further include those embodied in certain compositions generally described in above-referenced International Patent Application No. WO 02/03896.

Such systems still further include those embodied in certain compositions generally described in above-referenced International Patent Application No. WO 03/000224.

Such systems still further include those embodied in certain compositions generally described by Wang & Lee (2002), op. cit.

Such systems still further include those embodied in certain compositions generally described by Gavini et al. (2002), op. cit.

Such systems still further include those embodied in certain compositions generally described by Karasulu et al. (2002), op. cit.

Such systems still further include those embodied in certain compositions generally described in above-referenced U.S. Patent Application Publication No. 2003/0091540.

Such systems still further include those embodied in certain compositions generally described in above-referenced U.S. Patent Application Publication No. 200310219472.

Such systems still further include those embodied in certain compositions generally described in above-referenced U.S. Patent Application Publication No. 2004/0151774.

Bioadhesion, for example to a vaginal mucosal surface, is an important property of compositions of the invention, It is believed, without being bound by theory, that bioadhesion allows for a sustained and controlled delivery of at least the second active agent over time. Advantages over conventional vaginal delivery systems exhibiting less or no bioadhesion include one or more of:
(a) minimization of leakage of the composition from the site of application;
(b) suitability for application at any time of day, not limited to bedtime;
(c) reduction of active agent exposure, in particular systemic exposure, during a course of therapy;
(d) reduction of total active agent dose giving an acceptable clinical response;
(e) continuous active agent release during an extended period;
(f) more rapid relief of symptoms; and
(g) potential for single-dose therapy.

At least a component of the composition exhibits the property of bioadhesion. In one embodiment the composition comprises a non-bioadhesive component for delivery of the first active agent and a bioadhesive component for delivery of the second active agent. This embodiment can be useful where, for example, it is desired to deliver the first active agent as a bolus but permit delivery of the second active agent over a prolonged period of time. More typically, however, bioadhesion is a property of the composition as a whole.

Bioadhesion can be promoted by inclusion in the composition of one or a combination of bioadhesive, or more specifically mucoadhesive, agents that can independently be natural or synthetic; anionic, cationic or nonionic; and water-soluble or water-insoluble. In one embodiment the composition comprises a water-insoluble but water-swellable polymer capable of forming hydrogen bonds. Typically such a polymer is cross-linked and has a molecular weight of about 500 to about 3000 kDa, for example about 1000 to about 2000 kDa. Particular examples include, without limitation, polycarboxylic acid based polymers such as poly(acrylic, maleic, itaconic, citraconic, methacrylic, hydroxyethyl-methoxyethyl- and methoxyethoxyethylmethacrylic) acids and derivatives thereof including salts and esters. Such polymers illustratively include acrylate/methacrylate copolymers with quaternary ammonium functional groups, and ethylacrylate/methylmethacrylate copolymers with natural ester groups, as available for example under the Eudragit® brand. Another example is polycarbophil, which is a polyacrylic acid cross-linked with divinyl glycol. Alternative bioadhesive agents include cellulose derivatives such as methyl-, ethyl-, methylethyl-, hydroxymethyl-, hydroxy-ethyl-, hydroxypropyl-, hydroxyethylethyl-, carboxymethyl- and hydroxypropylmethyl-celluloses, and esters, ethers and salts thereof; gums such as acacia, xanthan, guar, locust bean, tragacanth, karaya, ghatti, cholla and psyllium seed gun-is and gum arabic; clays such as montmorillonite and attapulgite; polysaccharides such as dextrans, pectins, amylopectins, agars, carrageenans, mannans, scleroglucans, polygalactonic acids, starches and starch derivatives, e.g., hydroxypropyl starch and carboxymethyl starch; lipophilic preparations containing polysaccharides such as Orabase®; carbohydrates polysubstituted with groups such as sulfate, phosphate, sulfonate or phosphonate groups, e.g., sucrose octasulfate; polypeptides such as casein, gluten, gelatin and fibrin glue; chitosan or salts or derivatives thereof including chitosan chloride, chitosan lactate and chitosan glutamate; carboxymethyl chitin; glycosaminoglycans such as hyaluronic acid; alginic acid or salts thereof including sodium and magnesium alginates; adhesives containing bismuth oxide or aluminum oxide; atherocollagen; polyvinyl polymers such as polyvinyl alcohols, polyvinyl methylethers, polyvinylpyrrolidone and polycarboxylated vinyl polymers; polysiloxanes; polyethers; polyalkylene (e.g., polyethylene) oxides and glycols; polyalkoxy and polyacrylamide polymers and derivatives and salts thereof, polyglycolic and polylactic acid homopolymers and copolymers; glycolide/lactide copolymers, e.g., poly-L-(lactide coglycolide); and glyceryl monooleate. Further information on these and other bioadhesive agents that may be useful herein can be found in above-referenced International Patent Publication No. WO 2005/087270. Particular agents mentioned therein as causing minimal irritation and not affecting the normal vaginal flora include polyacrylic hydrogels, polyvinyl alcohol, hydroxypropylcellulose, hydroxypropylmethyl-cellulose, xanthan gum and chitosan.

Bioadhesion can also be provided using an in situ gelling polymer system such as a thermoreversibly gelling copolymer in combination with a bioadhesive agent, for example as described in above-referenced U.S. Patent Application Publication No. 2003/0091642.

The particular form of a composition useful herein is not limited and can be, for example, a cream, a gel, a foam, a vaginal tablet, pessary or suppository, a tampon, an implant such as a ring, etc.

However, of particular interest herein is a composition in the form of a water-in-oil emulsion as generally described in any of above-referenced U.S. Pat. No. 4,551,148, U.S. Pat. No. 5,055,303, U.S. Pat. No. 5,266,329 or U.S. Patent Application Publication No. 2003/0180366, or as farther described herein. Such a water-in-oil emulsion can be presented in a solid form, for example as a vaginal suppository, or in a semi-solid form, for example as a vaginal cream, and has bioadhesive properties.

In one embodiment, a composition of the invention has an external lipoidal phase and an internal nonlipoidal phase, and can provide for example a bioadhesive vaginal delivery system as described by Thompson & Levinson (2002), op. cit., or a vaginal delivery system substantially equivalent thereto. The bioadhesive property of such a composition is believed, without being bound by theory, to reside at least in part in the lipoidal nature of the external phase, which repels moisture and thereby resists dilution and removal by normal vaginal secretion. It is further believed, again without being bound by theory, that the lipoidal external phase serves to sequester the internal nonlipoidal phase; in embodiments wherein one or more active agents are present partly or wholly in the internal phase, the active agent payload is likewise sequestered, allowing for release of the active agent(s) to be metered slowly over time.

The bioadhesive and controlled or sustained release properties of a composition embodying a vaginal delivery system known as the Site Release® (SR) system useful herein have been demonstrated in studies summarized by Merabet et al. (2005), Expert Opin. Drug Deliv. 2(4):769-777, incorporated herein by reference but not admitted to be prior art to the present invention.

A “conventional” vaginal cream herein refers to a semi-solid emulsion having a continuous aqueous or nonlipoidal phase and a discontinuous or disperse nonaqueous or lipoidal phase, i.e., an oil-in-water emulsion, wherein an active agent is solubilized or dispersed in the continuous phase. Such a cream, while “conventional” in its general structure, can nonetheless represent an embodiment of the present invention if it has bioadhesive properties and contains at least two active agents formulated in such a way as to exhibit different release profiles as required herein.

However, solubilization or dispersion of an active agent in the continuous nonlipoidal phase of an oil-in-water emulsion permits immediate contact of the active agent with the vulvovaginal surface to which the composition is applied, but also permits dilution, rinsing and leakage of the composition from this surface, reducing the contact time with the surface and, where the active agent is an anti-infective agent, with the targeted pathogens. An oil-in-water emulsion comprising, for example, an antibacterial agent and/or an antifungal agent therefore must generally be administered repeatedly, for example about 3 to 7 times a week, to provide a clinically acceptable response. Such repeated application increases the potential for systemic delivery of the active agent, and thereby increases the potential for adverse side-effects, and also increases likelihood of tissue irritation. For these reasons, water-in-oil emulsion formulations such as the SR system are generally preferred herein.

Weinstein et al. (1994), Clin. Ther. 16(6):930-934, studied the retention time of vaginal creams containing 2% butoconazole nitrate. A total of 16 healthy women were treated intravaginally with a conventional vaginal cream or a bioadhesive SR cream, and monitored daily over 7 days for the amount of residual cream detected within the vaginal cavity by gynecological swab. A median retention time of 4.2 days was reported for the SR cream, by comparison with about 2.5 days for the standard cream.

Thompson & Levinson (2002), op. cit., reported a study in which 28 healthy women received intravaginal treatment with a conventional antifungal vaginal cream or a bioadhesive SR cream containing the same antifungal agent, in either case as a single dose. The women wore mini-pads for a 48-hour period to evaluate product leakage from the vaginal cavity. At each time point studied (3, 6, 24 and 48 hours after administration), product leakage was reportedly greater with the conventional cream than with the SR cream. Overall, leakage was reduced by over 50% with the SR cream.

Conventional vaginal creams commonly require application at bedtime to take advantage of a supine position of the patient for several hours, which can help to retain the cream within the vaginal cavity. The bioadhesive property and consequently enhanced vaginal retention of a vaginal cream of the invention can enable application at any convenient time of day.

Thompson & Levinson (2002), op. cit., also reported in vitro analysis of butoconazole nitrate release properties of a conventional vaginal cream and a cream embodying the SR system, using a pH 4.3 acetate buffer, designed to simulate vaginal fluid. The conventional cream was reported to disintegrate rapidly and begin to release the active agent immediately, with substantially all of the active agent payload being released within 1 to 4 hours. By contrast, the SR cream was reported to release the active agent continuously over about 7 days.

The bioadhesive and sustained release properties of a water-in-oil vaginal cream illustrative of the invention, e.g., an SR cream, can permit a relatively low dose of an active agent to provide a clinically acceptable response at least substantially equal to that provided by a much larger dose of the active agent administered in the form of a conventional cream. In particular, a single administration of an SR cream can provide a clinically acceptable response at least substantially equal to that provided by a conventional cream administered more than once, for example repeatedly about 3 to about 7 times in the course of one week. In this regard it is noted that adverse drug reactions are generally dose related, with appearance of new adverse events or exacerbation of existing adverse effects as the dose is escalated. An SR composition therefore has the potential to provide an improved safety profile. This is especially true with respect to adverse effects resulting from systemic delivery. The drug-sparing effect of a sustained release profile permitted by preferred compositions tends to reduce systemic delivery yet still provides therapeutically effective delivery at the locus of administration.

A composition of one embodiment of the invention typically comprises a multiplicity of unit cells, which are the basic repeating units of the delivery system and are not divisible without losing at least some of the properties useful herein. Each unit cell has internal and external phases, corresponding to the internal and external phases of the composition referred to above. Compositions having such multiphase structure can be described using conventional classifications, for example as emulsions, emulsion/dispersions, double emulsions, suspensions within emulsions, suppositories, foams, creams, ovules, inserts, and so on. Preferred compositions of the invention are in the form of water-in-oil emulsions having medium to high internal phase ratio (expressed as percentage of total volume occupied by the internal phase), for example greater than about 60%, greater than about 70%, or greater than about 75%, by volume.

Compositions useful herein include liquids or semi-solids having a viscosity of about 5,000 to about 1,000,000 centipoise, for example about 100,000 to about 800,000 centipoise. In certain embodiments the composition is a vaginal cream having a viscosity of about 5,000 to about 750,000 centipoise, for example about 350,000 to about 550,000 centipoise. A vaginal cream is generally a semi-solid water-in-oil emulsion and comprises an emulsifying agent. It is believed, without being bound by theory, that bioadherence of the composition to the vulvovaginal surface, for example the vaginal mucosal surface, requires that the composition have sufficient viscosity to retain its integrity when applied to such a surface. Optional ingredients that can increase viscosity, among other properties, include microcrystalline wax, colloidal silicon dioxide, and various pharmaceutically acceptable polymers including polysaccharides, cellulosic polymers such as carboxymethylcellulose, methylcellulose, hydroxypropylmethylcellulose, etc., polyethylene glycol, acrylate polymers and the like.

Solid compositions comprising a water-in-oil emulsion typically melt at body temperature to form a bioadhesive cream substantially as described above.

In preferred compositions, the internal phase is typically discontinuous and, as indicated above, is nonlipoidal. The nonlipoidal character of the internal phase renders it miscible with water. Illustratively, the internal phase comprises water, glycerin, propylene glycol, sorbitol or a combination of two or more thereof. Generally the internal phase has high osmotic pressure. The internal phase can itself be monophasic, biphasic or multiphasic, taking the form, for example, of a solution, suspension, emulsion or combination thereof. The internal phase optionally comprises one or more suspended solids, emulsifying and/or dispersing agents, osmotic enhancers, extenders, diluents, buffering agents, chelating agents, preservatives, fragrances, colors, or other materials.

Optionally, the internal phase is acid buffered to an internal pH of about 2.0 to about 6.0, for example about 2.5 to about 5.5 or about 3.5 to about 5.0. In one embodiment the internal phase is acid buffered to an internal pH that is substantially optimal to the vaginal environment, i.e., a pH that does not cause substantial irritation, itching or other discomfort and/or renders the vaginal environment less hospitable to common pathogens including fungal and bacterial pathogens. Typically such a pH is about 4.0 to about 5.0, for example approximately 4.5.

The external phase of preferred compositions is typically continuous (in such systems adjacent unit cells have common external phases) and, as indicated above, is lipoidal. The term “lipoidal” herein can pertain to any of a group of organic compounds including neutral fats, fatty acids, waxes, phosphatides, petrolatum, fatty acid esters of monoprotic alcohols, mineral oils, etc., having the following properties: insoluble in water; soluble in alcohol, ether, chloroform or other fat solvents; and exhibiting a greasy feel. Examples of suitable oils are mineral oils having viscosity of about 5.6 to about 68.7 centistokes, for example about 25 to about 65 centistokes, and vegetable oils such as coconut, palm kernel, cocoa butter, cottonseed, peanut, olive, palm, sunflower, sesame, corn, safflower, rapeseed (canola) and soybean oils and fractionated liquid triglycerides of naturally derived short-chain fatty acids.

The term “lipoidal” can also pertain to amphiphilic compounds, including for example natural and synthetic phospholipids. Suitable phospholipids can include, for example phosphatidylcholine esters such as dioleoylphosphatidylcholine, dimyristoyl-phosphatidylcholine, dipentadecanoylphosphatidylcholine, dipalmitoylphosphatidyl-choline (DPPC) and distearoylphosphatidylcholine (DSPC); phosphatidylethanolamine esters such as dioleoylphosphatidylethanolamine and dipalmitoylphosphatidylethanol-amine (DPPE); phosphatidylserine; phosphatidylglycerol; phosphatidylinositol; etc.

In one embodiment, the external phase comprises a phospholipid component, for example a lecithin component, more particularly a refined lecithin component. Without being bound by theory, it is believed that refined lecithins or other phospholipid materials can reside at the oil-water interface of a water-in-oil emulsion and impart improved stability to the emulsion, especially where an active agent is present having surfactant properties that tend to disrupt emulsion stability. A preferred lecithin comprises not less than about 70%, for example not less than about 80%, phosphatidylcholine. The phosphatidylcholine content of the lecithin can be as high as about 96% or even higher. Food grade lecithin may or may not be found acceptable in specific formulations. An example of a refined lecithin that is generally suitable is Phospholipon 90™, available from American Lecithin Co.

Amphiphilic compounds other than phospholipids can also act, optionally together with a phospholipid, as emulsifying agents in a composition of the invention. Any pharmaceutically acceptable emulsifying agent or combination thereof can be used, including without limitation medium and long chain monoglycerides and diglycerides, such as glyceryl monooleate, glyceryl monostearate, glyceryl monoisostearate and glyceryl monopalmitate, polyglyceryl esters of fatty acids, such as polyglyceryl-3 oleate, and polyethylene glycol esters and diesters of fatty acids, such as PEG-30 dipolyhydroxystearate. Such agents can also function as emollients in the composition. Emulsifying agents soluble in the external phase are generally preferred. In one embodiment a mono- and diglyceride mixture is used, alone or with addition of a metallic soap such as aluminum stearate.

Water-in-oil emulsion compositions of the invention are typically deformable at physiological temperatures (approximately 37° C.) but, unlike conventional creams, do not rapidly lose integrity upon application to a vaginal mucosal surface. In general, therefore, they do not result in offensive or otherwise unacceptable leakage from the vaginal cavity following administration. As physical breakdown of such compositions occurs over an extended period, nonaqueous components are either absorbed or released from the vaginal cavity at a generally unnoticeable rate, making no substantial increase over normal rates of vaginal secretion.

The first or second active agent or both can be present in either one or both of the internal and external phases of a water-in-oil emulsion composition of the invention. However, two embodiments are of particular interest.

In one of these two embodiments, the first active agent is predominantly to substantially contained in the external phase and the second active agent is predominantly to substantially contained in the internal phase. The term “predominantly contained” in the present context means that more than about 50%, for example more than about 75%, by weight of the agent is present in the phase in question. The term “substantially contained” in the present context means that any amount of the agent that is present elsewhere than in the phase in question contributes negligibly in practical terms to the overall delivery dynamics of the composition. It will be understood that through normal physical processes such as diffusion some portion of an agent “substantially contained” in one phase of the composition can be expected to transfer to the other phase. However, such transfer is normally inconsequential. Typically, in the present embodiment, at least about 85%, for example at least about 90% or at least about 95% and in some cases at least about 98% or even at least about 99%, of the first active agent is present in the external phase and at least about 85%, for example at least about 90% or at least about 95% and in some cases at least about 98% or even at least about 99%, of the second active agent is present in the internal phase.

In this embodiment it is believed, without being bound by theory, that location of the first active agent in the external phase typically permits substantially immediate delivery of the first active agent into the vaginal mucosa or other tissue to which the composition is administered. This can be useful where a rapid therapeutic effect is desired, for example in relief of symptoms. Meanwhile, location of the second active agent in the internal phase typically permits delivery of the second active agent to be delayed or slowed (and therefore sustained or extended) relative to delivery of the first active agent.

In another embodiment both agents are present at least in part in the internal phase of the composition, and can be in dispersed form, for example in solution or suspension therein, or in non-dispersed form. Optionally, the first and/or the second active agents are predominantly to substantially contained in the internal phase. The first active agent is present in a form adapted for release over a relatively short period and the second active agent is present in a form adapted for delayed release and/or for release over a relatively long period.

Differential release of the active agents in a composition of this embodiment can be secured by investing the second active agent with one or more release-controlling means effective to provide a release profile that is substantially delayed and/or extended relative to the release profile of the first active agent. Such means include without limitation (a) formulating the second active agent in solid particulate form but solubilizing the first active agent; (b) formulating both agents in solid particulate form but providing a particle size for the second active agent; (c) at least partially encapsulating the second active agent in a barrier layer that retards and/or slows release; (d) at least partially segregating the first and second active agents in different internal phases such that the medium surrounding the first active agent promotes more rapid release than that surrounding the second active agent; (e) formulating at least the second active agent in solid lipid nanoparticles as generally described, for example, in above-referenced International Patent Publication No. WO 2005/087270; and (f) means substantially equivalent to any of these.

Solubilization of one or both agents can be achieved, for example, by use of a cosolvent and/or surfactant. Some agents, for example the antibacterial agent metronidazole, are readily water soluble or easily solubilized, and such agents are typically present at least in substantial part in solution in the internal phase. Commonly, however, one or both agents can be present at least in part in particulate form, for example in micronized form or in nanoparticulate form, and can be dispersed as a particulate suspension in the internal and/or external phase. In various embodiments the first or second active agent or both are present in aggregates or liposomes within the internal and/or external phase.

In compositions having one or both active agents in solid particulate form, any suitable particle size can be used, and, as indicated above, a different range of particle size can be selected for each agent (wherein typically the second active agent is formulated at larger particle size). Typically, however, good physical stability may be difficult to achieve where a substantial portion of the particles of either agent are greater than about 250 μm in diameter. Thus a D₉₀ particle size (wherein 90% by weight of the particles are smaller than the specified size) not greater than about 250 μm is generally desirable for both agents. Preferably at least 99% by weight of the particles are not greater than about 250 μm in diameter.

Particle sizes smaller than about 5 μm can be useful but the expense of particle size reduction may not be justified by any improvement in stability or efficacy at such particle sizes. Nonetheless, particle sizes as small as 0.4 μm (400 nm), or even as small as 50 nm, can be used if desired.

Release of the active agents from a composition of the invention can occur by one or more mechanisms, none of which is limiting to the present invention. Such mechanisms can include diffusion, for example from the internal phase through the external phase into the vaginal mucosa; rupture of unit cells; dissolution of solid particulates; etc. Release dynamics can be linear or nonlinear.

Compositional factors affecting release rate of each active agent can include relative amounts of the active agent present in the internal and external phases; internal phase ratio; osmotic pressure of the internal phase; pH of the internal phase; selection and relative amounts of lipoidal compounds, including amphiphilic compounds, in the external phase, influencing diffusibility of each active agent therein; particle size where the active agent is in solid particulate form; viscosity of the composition; etc. Each of these factors can be routinely modified by one of skill in the art based on the disclosure herein, to optimize release rate for specific situations. In a composition having at least the second active agent in the internal phase, and having a relatively small internal phase ratio, the external phase tends to form a relatively thick membrane through which the second active agent must pass to be released; accordingly release rate can be significantly slowed in such a composition. A composition wherein the unit cells are robust, i.e., resistant to rupture at least for a substantial portion of the period of bioadhesion, tends to exhibit diffusion-controlled release kinetics, whereas a composition wherein release occurs both by diffusion and unit cell rupture tends to exhibit more complex release kinetics.

Physiological factors affecting release rate of each active agent include factors affecting rate of physical breakdown or loss of integrity of the composition, such as amount and chemical nature of fluids and enzymes, pH, chemical balance, temperature and shear forces arising from body movement. Shear forces are believed not to affect integrity of water-in-oil compositions as rapidly or severely as in the case of conventional vaginal creams.

The composition is typically adapted to release the first active agent, the second active agent or both over a period of about 3 hours to about 10 days, upon application to a vulvovaginal surface, for example a vaginal mucosal surface. Based on the disclosure herein, including disclosure of documents incorporated by reference herein, in particular above-referenced U.S. Pat. Nos. 4,551,148 and 5,266,329 and U.S. Patent Application Publication No. 2003/0180366, as well as U.S. Patent Application Publication No. 2005/0095245, incorporated herein by reference but not admitted to be prior art to the present invention, one of skill in the art can without undue experimentation adjust release rate of each active agent from the composition to achieve a release period of about 3 hours to about 10 days. In various embodiments, the release period of at least one of the active agents is one of about 12 hours to about 10 days, about 1 to about 10 days, about 2 to about 10 days or about 3 to about 7 days.

A “release period” or equivalent phrase herein refers to a period during which the active agent is made available for absorption and pharmacological (e.g, antibacterial or antifungal) effect, such effect typically occurring at or close to the site of absorption, for example the vaginal cavity. Thus the “release period” begins when release substantially begins (e.g., immediately to about 1 hour after administration, or later in the case of the second active agent where this is formulated for delayed release), and ends when substantially no further active agent is available for release (e.g., about 3 hours to about 10 days after the beginning of the release period).

In one embodiment, the second active agent exhibits a release period that overlaps at least a terminal portion of the release period of the first active agent. In such a case the release profile of the second active agent can be a delayed-release profile (i.e., the beginning of the release period for the second active agent is substantially later than the beginning of the release period for the first active agent), an extended-release profile (i.e., the release period for the second active agent is substantially longer in duration than that for the first active agent), or both.

In another embodiment, the second active agent exhibits a release period that begins after release of the first active agent has peaked. In this case the second active agent exhibits delayed release and may or may not also exhibit extended release. In some cases, release of the second active agent can be delayed until after release of the first active agent is substantially complete, such that substantially no overlap in release periods of the first and second active agents occurs.

Differential release patterns of four non-limiting illustrative embodiments are shown diagrammatically in FIGS. 1-4. Other differential release patterns can be contemplated by one of skill in the art and are within the scope of the present invention.

In FIG. 1, the first active agent exhibits substantially immediate release and the second active agent exhibits delayed release. Neither active agent exhibits substantial properties of extended release. A differential release profile such as that illustrated in FIG. 1, wherein release of the second agent begins after release of the first agent has peaked, can be termed “tandem release”.

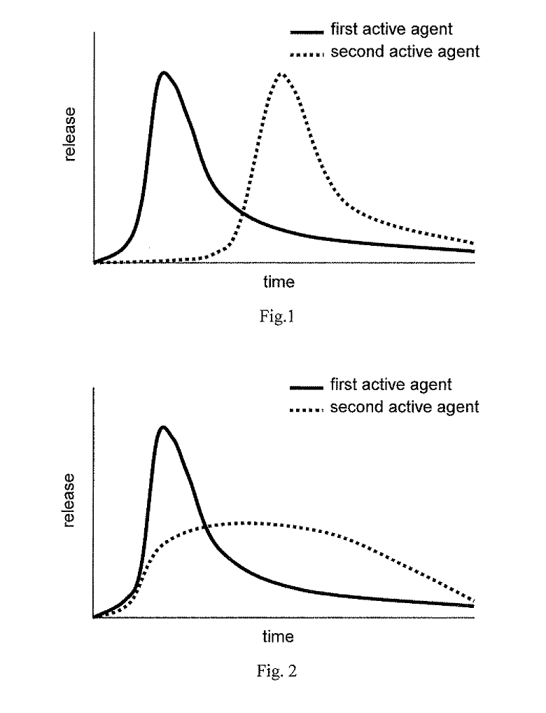

In FIG. 2, the first active agent exhibits substantially immediate release. The second active agent exhibits an extended release period that begins at about the same time as that for the first active agent (i.e., this is not a delayed-release profile) but continues substantially longer.

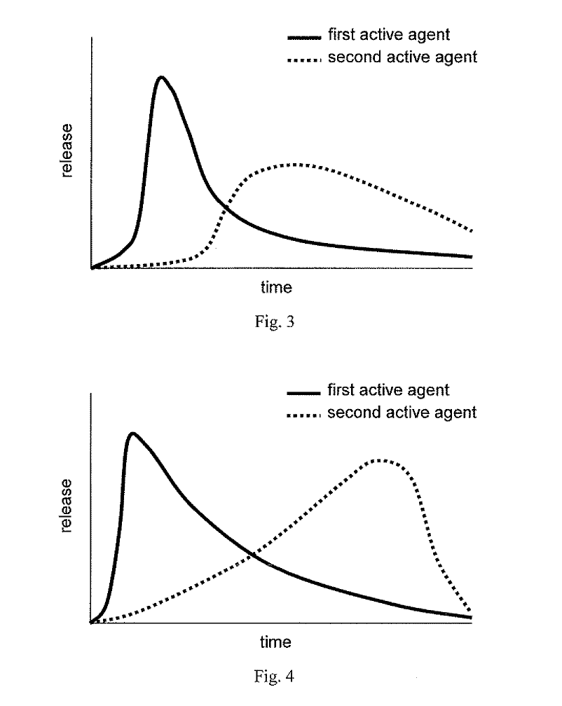

In FIG. 3, the first active agent exhibits substantially immediate release. The second active agent exhibits both delayed-release (later onset of release period) and extended-release (longer duration of release period) properties.

In FIG. 4, the first active agent exhibits a release profile characterized by a high rate of release early in the release period, and a declining rate of release thereafter. Meanwhile, the second active agent exhibits a release profile that is substantially inverted in relation to that of the first active agent, i.e., the release rate is initially slow and increases to a maximum later in the release period. A differential release profile such as that illustrated in FIG. 4 can be termed “inverse release”.

A wide range of release profiles is thus possible for each active agent. In one embodiment, at least one of the active agents exhibits, by 1 day after administration, about 2% to about 25% release; by 2 days after administration, about 15% to about 50% release; by 3 days after administration, about 25% to about 75% release; and by 4 days after administration, about 45% to 100% release.

Release rate can be determined by in vivo testing or by any suitable in vitro method. An illustrative in vitro method utilizes an open chamber diffusion cell system such as a Franz cell system, typically fitted with an appropriate inert synthetic membrane such as polysulfone, cellulose acetate/nitrate mixed ester or polytetrafluoroethylene of suitable thickness, e.g., 70 μm. The receptor medium should be one in which the active agent of interest is soluble, for example a water/ethanol medium. A test composition is placed uniformly on the membrane (illustratively, about 300 mg of a semi-solid composition such as a cream is a suitable amount for placement on a 25 mm diameter membrane) and is kept occluded to prevent solvent evaporation and compositional changes. This corresponds to an infinite dose condition. An aliquot of the receptor fluid is removed for analysis at appropriate intervals, and is replaced with an aliquot of fresh receptor fluid, so that the membrane remains in contact with the receptor fluid throughout the period of the release study. A release rate study such as that outlined above is typically replicated and can be conducted using a standard composition having known release properties for comparison.

The selection of first and second active agents is not limited, but illustratively they can be independently selected from anti-infectives, anti-inflammatories, analgesics, muscle relaxants, anesthetics, hormones, immunomodulators (including cytokine inhibitors and antihistamines) and antineoplastics, it being recognized that a drug can often be classified in more than one such category of active agent. More than two active agents can optionally be present.

In one embodiment, the first and second active agents comprise (a) an analgesic, muscle relaxant or anesthetic and (b) an immunomodulator. It will generally be found preferable to provide the analgesic, muscle relaxant or anesthetic as the first active agent and the immunomodulator as the second active agent, but these can be reversed if desired.

According to this embodiment, the analgesic, muscle relaxant or anesthetic can comprise, for example, an opioid analgesic, a non-opioid analgesic (e.g., a nonsteroidal anti-inflammatory drug or NSAID), a muscle relaxant or a local anesthetic.

Non-limiting examples of opioid analgesics include butorphanol, codeine, dihydrocodeine, fentanyl, hydrocodone, levorphanol, meperidine, methadone, morphine, naloxone, oxycodone, oxymorphone, pentazocine, propoxyphene, pharmaceutically acceptable salts and esters thereof and combinations thereof. Illustratively, fentanyl can be included in a composition of the invention in an amount of about 0.25% to about 10%, for example about 0.5% to about 5%, or about 1% to about 3%, by weight.

Non-limiting examples of non-opioid analgesics include capsaicin, diclofenac, salsalate, tramadol, pharmaceutically acceptable salts and esters thereof, and combinations thereof. Illustratively, capsaicin can be included in a composition of the invention in an amount of about 0.01% to about 10%, for example about 0.1% to about 10%, about 0.5% to about 5%, or about 1% to about 3%, by weight. Illustratively, diclofenac or tramadol can be included in a composition of the invention in an amount of about 0.25% to about 10%, for example about 0.5% to about 5%, or about 1% to about 3%, by weight.

Non-limiting examples of muscle relaxants include carisoprodol, methocarbamol, orphenadrine, pharmaceutically acceptable salts and esters thereof, and combinations thereof.

Non-limiting examples of local anesthetics include benzocaine, bupivacaine, butamben, chloroprocaine, cocaine, dibucaine, dyclonine, etidocaine, lidocaine, mepivacaine, pramoxine, prilocaine, procaine, proparacaine, ropivacaine, tetracaine, pharmaceutically acceptable salts and esters thereof, and combinations thereof. Illustratively, benzocaine, lidocaine or tetracaine can be included in a composition of the invention in an amount of about 0.25% to about 10%, for example about 0.5% to about 5%, or about 1% to about 3%, by weight.

According to the present embodiment, the immunomodulator can comprise, for example, an H₁, H₂ or H₃ receptor antagonist.

Non-limiting examples of H₁ receptor antagonists include acrivastine, azelastine, cetirizine, chlorpheniramine, cyproheptadine, desloratadine, diphenhydramine, ebastine, fexofenadine, hydroxyzine, ketotifen, levocabastine, loratadine, mizolastine, promethazine, pharmaceutically acceptable salts and esters thereof, and combinations thereof. Illustratively, diphenhydramine can be included in a composition of the invention in an amount of about 0.01% to about 10%, for example about 0.1% to about 10%, about 0.5% to about 5%, or about 1% to about 3%, by weight.

Non-limiting examples of H₂ receptor antagonists include burimamide, cimetidine, famotidine, nizatidine, ranitidine, pharmaceutically acceptable salts and esters thereof, and combinations thereof.

Non-limiting examples of H₃ receptor antagonists include betahistine, ciproxifan, GT-2331, iodoproxyfan, thioperamide, pharmaceutically acceptable salts and esters thereof, and combinations thereof.

In a further embodiment, the first and second active agents are anti-infectives, optionally in combination with at least a third active agent that can be another anti-infective or an agent of another class, for example an anti-inflammatory or a hormone such as an estrogen. Anti-infectives include antiviral, antibacterial, antifungal and antiprotozoal agents, it being recognized that some drugs have a spectrum of activity that runs across microbial types. The first and second active agents can both belong to one class of anti-infective, for example both can be antibacterial agents, e.g., having a complementary spectrum of activity. Alternatively, the first and second active agents can belong to different classes of anti-infective, for example an antibacterial and an antifungal.

Where a composition comprises as active agents an antibacterial and an antifungal, either one can be the first agent herein, the other being the second agent herein. Depending on circumstances, it can be advantageous to have the antibacterial release earlier or faster than the antifungal or vice versa. For example, for treatment of BV in a patient at risk of developing a fungal (e.g., Candida alticans) infection, a composition having an antibacterial as the first active agent and an antifungal as the second active agent can be useful. On the other hand, for treatment of VVC in a patient at risk of developing a bacterial (e.g., Gardnerella vaginalis) infection, a composition having all antifungal as the first active agent and an antibacterial as the second active agent can be useful. The present embodiment is more particularly illustrated herein with reference to a composition having an antibacterial as the first and an antifungal as the second active agent, but it will be understood that these can be reversed if desired.

An antibacterial agent useful as the first or second active agent herein, but in a particular embodiment the first active agent, can comprise any antibacterial kmown in the art to be useful in treatment of bacterial infections of the vulvovaginal system. The antibacterial can be one predominantly targeting a particular category of pathogenic bacteria, for example aerobic, anaerobic, gram-negative, gram-positive, etc. Illustrative examples of antibacterials that can be useful include without limitation acriflavine, ampicillin, ceftriaxone, chloramphenicol, chlorquinaldol, clindamycin, iodoquinol, metronidazole, nimorazole, ornidazole, pivampicillin, secnidazole, spiramycin, tetracycline, tinidazole, pharmaceutically acceptable salts and esters thereof, combinations thereof and the like. In one embodiment the first active agent comprises or consists essentially of clindamycin or a pharmaceutically acceptable salt or ester thereof, for example clindamycin hydrochloride or clindamycin phosphate. In a particular embodiment the first active agent comprises or consists essentially of clindamycin phosphate. In another embodiment the first active agent comprises or consists essentially of metronidazole or a pharmaceutically acceptable salt or ester thereof. An antibacterial agent such as butoconazole or metronidazole is present in the composition in an antibacterially effective amount.

Amounts of clindamycin or a salt or ester thereof are expressed herein as clindamycin (free base) equivalent amounts unless the context demands otherwise. Any antibacterially effective amount of clindamycin or salt or ester thereof can be used, but typically in a vaginal cream preparation a clindamycin equivalent amount of about 0.5% to about 6% by weight, for example about 1% to about 3% by weight, will be found useful.

Any antibacterially effective amount of metronidazole can be used, but typically in a vaginal cream preparation a metronidazole amount of about 0.1% to about 4% by weight, for example about 0.5% to about 1.5% by weight, will be found useful.

An antifungal agent useful as the first or second active agent herein, but in a particular embodiment the second active agent, can comprise any antifungal known in the art to be useful in treatment of fungal, especially candidal, infections of the vulvovaginal system. Illustrative antifungal agents include without limitation atovaquone, griseofulvin, nystatin, polymyxin B, terbinafine, and imidazole and triazole compounds such as butoconazole, clotrimazole, econazole, fluconazole, isoconazole, itraconazole, ketoconazole, miconazole, oxiconazole, ravuconazole, saperconazole, sertaconazole, sulconazole, terconazole, tioconazole and voriconazole, pharmaceutically acceptable salts and esters thereof, combinations thereof and the like. In one embodiment the second active agent comprises or consists essentially of butoconazole or a pharmaceutically acceptable salt or ester thereof. In a particular embodiment the second active agent comprises or consists essentially of butoconazole nitrate. Al antifungal agent such as butoconazole is present in the composition in an antifingally effective amount.

Amounts of butoconazole or a salt or ester thereof are expressed herein as butoconazole nitrate equivalent amounts unless the context demands otherwise. Any antifungally effective amount of butoconazole or salt or ester thereof can be used, but typically in a vaginal cream preparation a butoconazole nitrate equivalent amount of about 0.5% to about 6% by weight, for example about 1% to about 3% by weight, will be found useful.

It will be recognized by one of skill in the art that the terms “antibacterial” or “antifungal”, applied to an active agent herein, are not necessarily mutually exclusive. A particular agent can exhibit, to some degree, both antifungal and antibacterial activity. Some agents, for example certain imidazoles including metronidazole, are utilized herein principally for their antibacterial activity, but also possess a useful degree of antifungal (including anticandidal), and in some cases antiprotozoal (including antitrichomonal) activity. Where such an agent is included in a composition of the invention as an antibacterial agent, some additional benefit is therefore possible in supplementing the activity of an antifungal agent (e.g., butoconazole) against a fungal pathogen such as C. albicans.

A particular example of a vaginal cream composition of the invention has at least one nonlipoidal internal phase and at least one lipoidal external phase that is bioadhesive to a vaginal mucosal surface. The composition comprises clindamycin phosphate in a clindarnycin equivalent amount of about 2% by weight and butoconazole nitrate in an amount of about 2% by weight, wherein the butoconazole nitrate exhibits a release profile that is substantially delayed, extended or inverted relative to the release profile of the clindamycin phosphate.

Another particular example of a vaginal cream composition of the invention has at least one nonlipoidal internal phase and at least one lipoidal external phase that is bioadhesive to a vaginal mucosal surface. The composition comprises metronidazole in an amount of about 0.75% by weight and butoconazole nitrate in an amount of about 2% by weight, wherein the butoconazole nitrate exhibits a release profile that is substantially delayed, extended or inverted relative to the release profile of the metronidazole.

In one embodiment, the antibacterial or first active agent, illustratively metronidazole, is predominantly to substantially contained in the internal phase and is substantially solubilized therein, and the antifungal or second active agent, illustratively butoconazole nitrate, is likewise predominantly to substantially contained in the internal phase but is substantially in particulate form and suspended therein. It is believed, without being bound by theory, tlat solubilization of the first active agent but not of the second active agent in this manner can be responsible at least in part for the differential release property of the composition.

Illustratively, excipienit ingredients in a vaginal cream composition of the invention can include water, sorbitol (e.g., in the form of a sorbitol solution), lecithin, at least one long chain monoglyceride, for example glyceryl monooleate, glyceryl monostearate, glyceryl monoisostearate or glyceryl monopalmitate, at least one polyglyceryl or polyethylene glycol fatty acid ester, for example polyglyceryl-3 oleate or PEG-30 dipolyhydroxystearate, a chelating agent, for example edetate disodium, at least one antimicrobial preservative, for example methylparaben and/or propylparaben, mineral oil and microcrystalline wax.

A unit dosage amount of a composition of the invention is an amount suitable for a single administration to a vulvovaginal surface, for example a vaginal mucosal surface, as described herein. Most conveniently for the patient, the composition is provided in unit dose aliquots, typically individually packaged, but this is not a requirement of the present invention. A convenient unit dose aliquot of a vaginal cream is an amount of about 1 to about 10 g, although greater or lesser amounts, for example as little as about 0.1 g or as much as about 25 g, can be used if desired. A particularly suitable unit dosage amount of a vaginal cream is about 3 to about 6 g, for example about 5 g. Where a unit dosage amount is smaller, it may be desirable to increase the concentration of active agents in the composition, and vice versa.

Conveniently, a unit dosage amount of a vaginal cream of the invention can be furnished in a prefilled container or applicator, for example an applicator similar to that used for Gynazole-1® vaginal cream of KV Pharmaceutical Co., St Louis, Mo.

A delivery system for at least two active agents comprising a vaginal cream composition of the invention, for example a disposable applicator, more particularly a disposable applicator prefilled with a unit dosage amount of the composition, is an embodiment of the invention.

A composition of the invention in the form of a vaginal cream can be prepared by known batch or continuous processes for preparing pharmaceutical creams. As in preparing conventional emulsions, shear force is applied to the components by use of a mixer, homogenizer, mill, impingement surface, ultrasound, shaking or vibration. However, unlike conventional emulsions, water-in-oil emulsions of the invention should normally be prepared using mixing shear at a relatively low level to prevent destruction of the emulsion by excess energy. Illustratively, the internal and external phases are first prepared separately. In a typical batch process, the internal phase is added to the external phase while mixing in a planetary-type or other suitable mixer until a stable emulsion is formed. Addition rates and mixing speeds can be adjusted to optimize formation and viscosity of the emulsion. In a typical continuous process, the external phase is introduced into a continuous mixer that comprises a plurality of impellers, until it reaches the level of the lowest impeller in the mixing chamber. The two phases are then simultaneously introduced through the bottom of the mixer in proper proportion as the impellers rotate to apply shear to the components. The finished emulsion emerges through the top of the mixer. Flow rate through the mixing chamber and mixing speed can be adjusted to optimize formation and viscosity of the emulsion.

A composition of the invention can be administered topically to external surfaces of the vulva and/or to surrounding areas of skin. In addition or alternatively, the composition can be administered intravaginally. In one embodiment, the composition is a vaginal cream and is administered intravaginally in a unit dosage amount as defined above to a vaginal mucosal surface.

A vaginal cream of the invention can be administered to contact a mucosal surface in the vaginal cavity by means, for example, of an applicator that is optionally pre-filled with a single unit dosage amount of the cream. With the patient in a supine position, the tip of the applicator can be gently inserted high in the vagina, for example in the posterior vaginal fornix, and the cream can be released through the tip by pushing on a plunger of the applicator.

The invention provides a method for treating a condition of the vulvovaginal system for which a combination of a first active agent and a second active agent is indicated. The method comprises administering a pharmaceutical composition as described herein to a vulvovaginal surface, for example a vaginal mucosal surface.

As one illustration, a method of the invention for treating vulvodynia, including dysesthetic vulvodynia and vulvar vestibulitis syndrome, comprises administering a pharmaceutical composition as described herein to a vulvovaginal surface, e.g., topically to external surfaces of the vulva and/or to surrounding areas of skin, or intravaginally, wherein the composition has as the first and second active agents (a) an analgesic, muscle relaxant or anesthetic, for example lidocaine, and (b) an immunomodulator, for example diphenhydramine. Typically the first agent comprises the analgesic, muscle relaxant or anesthetic and the second agent comprises the immunomodulator.

As a further illustration, a method of the invention for treating a BV or mixed BV/VVC infection comprises administering a pharmaceutical composition, for example a vaginal cream composition, as described herein to a vulvovaginal surface, for example a vaginal miucosal surface, wherein the composition has as the first active agent an antibacterial agent such as clindamycin, metronidazole or a pharmaceutically acceptable salt or ester thereof, and as the second active agent an antifungal agent such as butoconazole or a pharmaceutically acceptable salt or ester thereof. Such a method can also be used for treating a secondary condition arising from such an infection.

Without being bound by theory, it is believed that therapeutic effectiveness of this method derives at least in part from the differential release of the antibacterial and antifungal agents. The antibacterial agent, illustratively clindamycin phosphate or metronidazole, exhibits faster and/or earlier release tlhian the antifungal agent, providing effective control of the bacterial, e.g., Gardnerella vaginalis, infection. Release of the antifungal agent, illustratively butoconazole nitrate, is maximized somewhat later, providing effective control and limiting explosion of a fungal, e.g., Candida albicans, population that could otherwise occur upon removal of the bacterial infection. Such an explosion can arise from an existing mixed bacterial/fungal infection, from small fungal colonies present in the vulvovaginal area, or as a de novo fungal infection after elimination of bacterial competition.

Such a method can involve repeated administration of a unit dosage amount of the composition until a clinically acceptable response is obtained; however, it is an advantage of at least those compositions of the invention having bioadhesive and sustained release properties that a clinically acceptable response is often obtainable with a single administration. A method wherein a single administration of a unit dosage amount provides a clinically acceptable response is often known as a “one dose to cure” therapy, but it will be recognized that the term “cure” in the present context does not necessarily mean total or permanent removal of the infection or total or permanent relief from all symptoms.

A clinically acceptable response or “cure” herein can be illustratively evidenced by one or more of the following outcomes:
(a) resolution of all four clinical “Amsel criteria”, namely normal vaginal discharge, vaginal pH <4.7, <20% clue cells on wet mount, and negative “whiff” test, as described by Amsel et al. (1983), Am. J. Med. 74:14-22;
(b) a “Nugent score” <4 by the gram stain interpretation method of Nugent et al. (1991), J. Clin. Microbiol. 29:297-301; and
(c) a physician's negative answer to the question, “In your opinion, does the patient require additional treatment for BV/VVC at this time?”

In one embodiment, a therapeutic method using a water-in-oil bioadhesive composition of the invention provides, by a single administration, a “cure” rate at least substantially equal to that provided by about 3 to about 7 applications of a conventional vaginal cream composition, containing the same antibacterial and antifungal agents at the same concentration as the composition of the invention, in the course of one week.

A method of the invention, wherein the first and second active agents are an antibacterial and antifumgal respectively, can be used for treatment of any combination of bacterial and fungal infections present in the vulvovaginal system, including without limitation infections involving:
(a) fungi, more particularly yeasts, especially Candida spp. including one or more of C. albicans, C. dubliniensis, C. glabrata, C. keqir, C. krusei, C. lusitaniae, C. neoformans, C. parasilopsis and C. tropicalis, of which the most common is C. albicans; and
(b) bacteria, commonly a variety of species including one or more of Bacteroides spp., Gardnerella vaginalis, Mobiluncus spp., Mycoplasma hominis and Peptostreptococcus spp., most commonly with G. vaginalis predominating.

A further list of bacterial species identified in women with BV has been reported by Fredricks et al. (2005), N. Engl. J. Med. 353:1899-1911, incorporated herein by reference but not admitted to be prior art to the present invention.

All patents and publications cited herein are incorporated by reference into this application in their entirety.

The words “comprise”, “comprises”, and “comprising” are to be interpreted inclusively rather than exclusively.

What is claimed is:

1. A pharmaceutical composition comprising a first active agent and a second active agent, the composition (i) comprising a component adapted for bioadhesion to a vulvovaginal surface, and (ii) providing differential release of the active agents at said surface, wherein the second active agent exhibits a release profile that is substantially delayed, extended or inverted relative to the release profile of the first active agent.

2. The composition of claim 1, wherein the vulvovaginal surface to which the composition is adapted for bioadhesion is a vaginal mucosal surface.

3. The composition of claim 1, wherein the differential release of the active agents is substantially as shown in FIG. 1.

4. The composition of claim 1, wherein the differential release of the active agents is substantially as shown in FIG. 2.

5. The composition of claim 1, wherein the differential release of the active agents is substantially as shown in FIG. 3.

6. The composition of claim 1: wherein the differential release of the active agents is substantially as shown in FIG. 4.

7. The composition of claim 1, having at least one nonlipoidal internal phase and at least one lipoidal external phase that is bioadhesive to the vulvovaginal surface.

8. The composition of claim 7 that is in a form of a vaginal cream.

9. The composition of claim 7, wherein the first active agent is predominantly to substantially contained in the external phase and the second active agent is predominantly to substantially contained in the internal phase.

10. The composition of claim 7, wherein the first and second active agents are predominantly to substantially contained in the internal phase, the first active agent being present in a form adapted for release over a relatively short period and the second active agent being present in a form adapted for delayed release and/or for release over a relatively long period.

11. The composition of claim 10, wherein, upon application to a vaginal mucosal surface, the first active agent has a release period that begins substantially immediately and lasts for about 3 hours to about 5 days, and the second active agent has a release period that begins substantially immediately to about 5 days after application and continues until about 1 to about 7 days after the end of the release period of the first active agent.

12. The composition of claim 10, wherein at least the second active agent is in particulate form, having a substantially larger particle size than the first active agent.

13. The composition of claim 10, wherein the first active agent is substantially solubilized in the internal phase, and the second active agent is substantially in particulate form, suspended in the internal phase.

14. The composition of claim 10, wherein the second active agent is at least partially encapsulated in a barrier layer that retards and/or slows the rate of release of the second active agent.

15. The composition of claim 1, wherein the first and second active agents are independently selected from the group consisting of anti-infectives, anti-inflammatories, analgesics, muscle relaxants, anesthetics, hormones, immune modulators and antineoplastics.

16. The composition of claim 1, wherein the first active agent is an antibacterial agent and the second active agent is an antifungal agent.

17. The composition of claim 16, wherein the antibacterial agent comprises one or more compounds selected from the group consisting of acriflavine, ampicillin, ceftriaxone, chloramphenicol, chlorquinaldol, clindaamycin, iodoquinol, metronidazole, nimorazole, ornidazole, pivampicillin, secnidazole, spiramycin, tetracycline, tinidazole and pharmaceutically acceptable salts and esters thereof.

18. The composition of claim 16, wherein the antifungal agent comprises one or more compounds selected from the group consisting of atovaquone, butoconazole, clotrimazole, econazole, fluconazole, griseofulvin, isoconazole, itraconazole, ketoconazole, miconazole, nystatin, oxiconazole, polymyxin B, ravuconazole, saperconazole, sertaconazole, sulconazole, terbinafine, terconazole, tioconazole, voriconazole and pharmaceutically acceptable salts and esters thereof.

19. A vaginal drug delivery system comprising the composition of claim 8 and an applicator.

20. The delivery system of claim 19, wherein the applicator is disposable.

21. The delivery system of claim 19, wherein the applicator is prefilled with a unit dose amount of the composition.

22. The delivery system of claim 21, wherein the unit dose amount of the composition is about 1 to about 10 g.

23. The delivery system of claim 21, wherein the unit dose amount of the composition is about 3 to about 6 g.

24. A method for treating a condition of the vulvovaginal system for which a combination of a first active agent and a second active agent is indicated, the method comprising administering to a vulvovaginal surface a pharmaceutical composition tat comprises the first active agent and the second active agent, wherein the composition comprises a component that is bioadhesive to said surface, and wherein the second active agent exhibits a release profile that is substantially delayed and/or substantially extended relative to the release profile of the first active agent.

25. The method of claim 24, wherein the vulvovaginal surface to which the composition is administered is a vaginal mucosal surface.

26. The method of claim 25, wherein (a) the condition is a bacterial vaginosis or mixed bacterial vaginosis and vulvovaginal candidiasis infection, (b) the first active agent is an antibacterial agent, and (c) the second active agent is an anliifngal agent.

27. The method of claim 26, wherein the composition is a vaginal cream that comprises at least one nonlipoidal internal phase and at least one lipoidal external phase that is bioadhesive to the vaginal mucosal surface.

28. The method of claim 27 wherein the composition is applied in a single dosage amount effective to provide an acceptable clinical response.

29. The method of claim 28, wherein the single dosage amount is about 1 to about 10 g.
